(19) United States
(12) Patent Application Publication
(10) Pub. No.: US 2003/0088092 A1
(43) Pub. Date: May 8, 2003

(54) Water-soluble porphyrin derivatives and methods of their preparation

(75) Inventor: Nikolay E. Nifantiev, Moscow (RU)

Correspondence Address: BOLESH J. SKUTNIK PhD, JD, 515 Shaker Road, East Longmeadow, MA, 01028, US

(73) Assignee: CeramOptec Industries, Inc.

(21) Appl. No.: 10/306,046

(22) Filed: Nov. 27, 2002

Related U.S. Application Data
(63) Continuation-in-part of application No. 09/871,772, filed on Jun. 1, 2001.
(63) Continuation-in-part of application No. 10/151,764, filed on May 20, 2002.

(51) Int. Cl. C07D 487/22; C07H 5/04

(52) U.S. Cl. 540/145; 536/55.2

(57) ABSTRACT

The invention relates to the chemistry of biologically active compounds, namely, to a new method to prepare water-soluble porphyrin derivatives, particularly chlorin derivatives having general formulae (1). The compounds of the present invention are useful as photosensitizers, as drug substances and optionally in suitable drug products, for the photodynamic therapy of cancer, of infectious and other diseases, as well as for light irradiation treatments in other cases.

B is a ring having the structure:

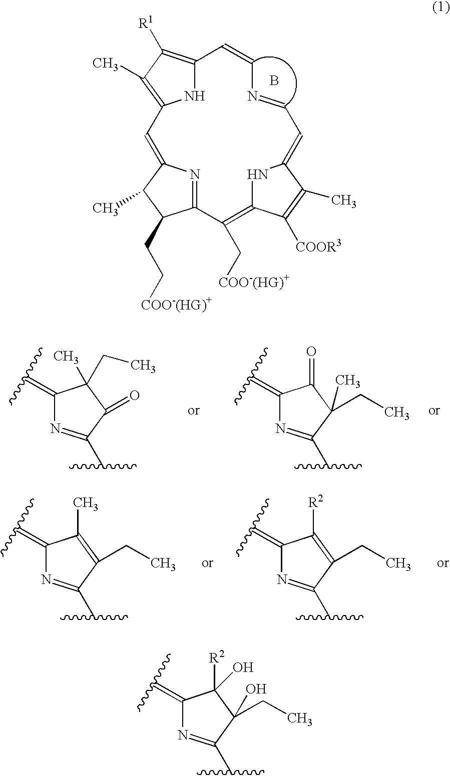

where:

R¹=—CH═CH₂, —CH(OAlk)CH₃, —CHO, —C(O)CH₃; —CH₂CH₃; —CH(Alk)CH(COAlk)₂, —CH₂CH(COAlk)₂, —CH(Alk)CH₂COAlk, —CH(Alk)CH₂CH(OH)CH₃, —CH₂CH₂CH(OH)CH₃

R²=—CH₃, —CHO, —CH(OH)Alk, —CH═CHAlk, CH₂OH, CH₂OAlk;

R³=H or lower alkyl;

G=hydrophilic organic amine (f.ex. N-methyl-D-glucamine and other amino-group containing carbohydrate derivatives, TRIS, aminoacids, oligopeptides).

Alk=alkyl substituent.

### REFERENCE TO RELATED CASE

This application is a continuation-in-part of co-pending U.S. patent application Ser. No. 09/871,772 filed on Jun. 1, 2001 by Nikolay E. Nifantiev, inventor, entitled “WATER SOLUBLE PORPHYRIN DERIVATIVES AND METHODS OF THEIR PREPARATION”, and of co-pending U.S. patent application Ser. No. 10/151,764 filed on May 20, 2002 by Nikolay E. Nifantiev and Dmitri V. Yashunsky, inventors, entitled “WATER SOLUBLE PORPHYRIN DERIVATIVES FOR PHOTODYNAMIC THERAPY, THEIR USE AND MANUFACTURE”, and incorporated by reference herein.

### FIELD OF THE INVENTION

The invention relates to the chemistry of biologically active compounds, namely, to a new method to prepare water-soluble porphyrin derivatives, particularly chlorin derivatives. The compounds of the present invention can be used as photosensitizers for the photodynamic therapy of cancer, infections and other diseases as well as for light irradiation treatments in other cases.

### BACKGROUND OF THE INVENTION

Photodynamic therapy (PDT) is one of the most promising new techniques now being explored for use in a variety of medical applications (Photodynamic therapy, basic principles and clinical applications. Eds. B. W. Henderson, Th. .J. Dougherty, Marcel Dekker, 1992, New York), and particularly is a well-recognized treatment for the destruction of tumors (Photodynamic tumor therapy. 2^{nd} and 3^{rd} generation photosensitizers. Ed. J. G. Moser, Harwood Academic Publishers, 1998, Amsterdam). PDT employs photosensitizers that are activated by radiation of a certain wavelength to oxidize and necrotize tissue. Because photosensitizers tend to preferentially accumulate around cancerous and hyperproliferative cells, they are particularly effective for necrotizing such tissue. Porphyrins are compounds widely used in PDT. The problem in pharmaceutical application of porphyrins is their low solubility in physiological solutions, rejecting the possibility to prepare injectable solutions for the PDT and for some other applications.

Methods to prepare water soluble porphyrin derivatives for PDT are known in the art. U.S. Pat. No. 5,330,741 by Smith discloses a method to prepare trisodium lysyl-chlorin p₆ involving the interaction between purpurin 18 methyl ester, resulting from methyl pheophorbide a transformation, and aqueous lysine in methylene chloride in the presence of pyridine. The mixture is stirred at room temperature for 12 hours, followed by the removal of the solvents in a high vacuum. The so prepared crude product is purified by the reversed-phase HPLC and subsequently lyophilized. To prepare an injectable solution for the PDT for cancer treatment, the preparation is dissolved in phosphate buffer solution, 0.1 N sodium hydroxide is added, the pH value of the solution being adjusted to pH 7.35 using 0.1 N HCl, followed by the sterility filtration through a microporous filter.

The drawbacks of the above mentioned method include bad reproducibility, hard work-up and utilization of toxic reagents. These limitations make this method inappropriate for pharmaceutical manufacturing. In addition, the prepared water soluble product of interest is stable in an aqueous solution for only 24 hours at 4° C. in the dark, and in solid form for up to 4 months at 4° C. in the dark [M. W. Leach, R. J. Higgins, J. E. Boggan, S.-J. Lee, S. Autry, K. M. Smith, Effectiveness of a Lysylchlorin p₆/Chlorin p₆ mixture in Photodynamic Therapy of the Subcutaneous 9 L Glioma in the Rat. Cancer Res., 1992, 52, 1235-1239; U.S. Pat. No. 5,330,741].

There is a method to prepare a water-soluble sodium pheophorbide a, described in U.S. Pat. No. 5,378,835 by Nakazato. According to the cited invention, pheophorbide a (2) is dissolved in diethyl ether, and a very diluted solution of alkali in n-propanol, iso-propanol or in their mixture is added dropwise and very slowly to the solution. The reaction is being undergone up to the complete precipitation of pheophorbide a salt, being separated by centrifugation and dried in vacuo. Then the product is dissolved in water resulting in a solution with concentration 0.5% and pH 9.2-9.5, that is then diluted with phosphate buffer with pH 7.4-7.8.

The drawback of the referred method is the fact that a concentrated (>1%) injectable pheophorbide a solution in water can not be generated by this technique. Additionally, the authors of the present invention demonstrated chemical instability of such salts when stored dryly, and their incomplete ability to dissolve in water after having been stored in the dry state.

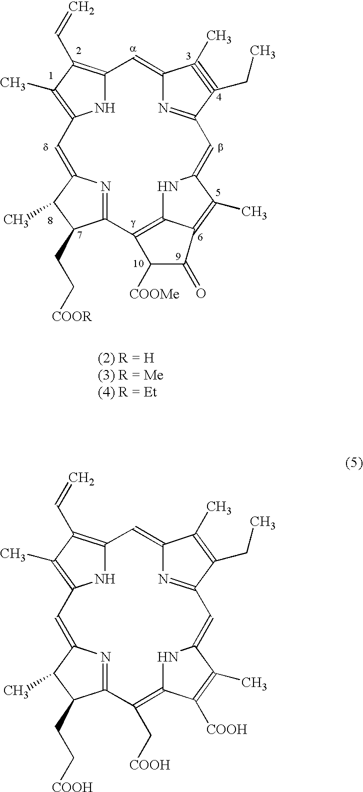

The closest analogue to the present invention is the method disclosed in Russian Patent No. RU2144538 by Ponomarev et al to prepare water-soluble complexes of chlorin e₆ with spacious organic amines including N-methyl-D-glucosamine by a series of straightforward sequences of chemical reactions including preparation of chlorophyll a from Spirulina Platensis cyanobacteria biomass, further conversion into chlorin e₆ according to standard procedures [S. Lötjönen, P. H. Hynninen, An improved method for the preparation of (10R)- and (10S)-pheophytins a and b. Synthesis. 1983, 705-708; P. H. Hynninen, S. Lötjönen, Preparation of phorbin derivatives from chlorophyll mixture utilizing the principle of selective hydrolysis. Synthesis. 1980, 539-541; S. Lötjönen, P. H. Hynninen, A convenient method for the preparation of wet chlorin e₆ and rhodin g₇ trimethyl esters. Synthesis, 1980, 541-543] with the overall yield exceeding 50% after precipitation of chlorin e₆ by way of stepwise addition of water to its acetone solution, separation by centrifugation and 3-fold washing with water and subsequent treatment of wet chlorin e₆ with water solution of 2 g-eq. spacious organic amine.

Key disadvantages of this method which cause critical difficulties for preparative syntheses of water-soluble chlorins and particularly for industrial syntheses and drug manufacturing are the following:

1. Chlorin e₆ as intermediate product is obtained as wet mass with unknown definite content of chlorin that brings uncertainties for standardization of its further solutions.

2. A key intermediate in the synthetic sequence is pheophorbide a (2) which is difficult to handle for purification and standardization due to its acidic properties. Separation of (2) via repeatable precipitations (as used by Ponomarev) is not quantitative and thus not convenient for large scale preparations.

3. Pheophorbide a (2) obtained by the indicated method contains impurities that are difficult to separate. This disadvantage causes uncertainty in quantification of (2) and disturb the chemical opening of cyclopentanone ring in the course of transformation of (2) into chlorins.

4. The samples of water soluble salts of chlorin e₆ being prepared according to Ponomarev contain a variety of impurities of non-porphyrin and porphyrin types which could not be separated from the target chlorin e₆ product with the use of the procedures described in the prior art. Particularly, among the porphyrin impurities one could find, by using TLC and HPLC methods, the pheophorbide a (2), purpurin 18 (6) and chlorin p6 (7) and some other contaminants.

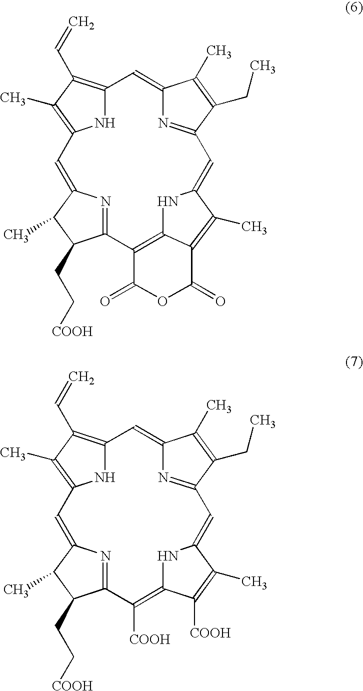

It should also be noted that the compounds of types (2), (6) and (7) as salts with hydrophilic amines of present inventions are characterized by remarkably lower water solubility as compared with respective chlorin e₆ salts. Nevertheless, in the presence of chlorin e₆ salts the compounds of types (2), (6) and (7) as salts with hydrophilic amines of present inventions are more water soluble than in isolation, which could be explained by the possible formation of complexes with chlorin e₆ salts. This phenomenon makes it impossible to separate the chlorin e₆ products from the impurities like the compounds of types (2), (6) and (7) by using the differences in their water solubilities.

5. The organic amines being used in the prototype invention for preparation of water-soluble chlorins are not optimal for practical applications. In particular, D-glucosamine, which forms complexes with chlorin having a higher water solubility, is not stable enough due to the possible oxidation at its aldehyde group. Furthermore, D-glucosamine can be present in the solution in several isomeric forms which creates structural uncertainties and corresponding difficulties for detailed structural characterization, thus failing to meet the demands of quality control for pharmaceutical preparations. One other spacious amine has been used in the prior art, namely, N-methyl-D-glucosamine which has the same disadvantages as the above mentioned D-glucosamine and, moreover, is hardly available due to its difficult preparation.

6. The prototype invention claims the formation of water-soluble salts of chlorine e₆ derivatives with spatial organic amines which is very uncertain because usual spatial organic amines, e.g. those ones containing tert-butyl, neopentyl, adamantyl, cyclohexyl groups, could not be used in the preparation of water-soluble chlorin e₆ salts due to due to high hydrophobicity of spatial organic moieties.

These circumstances make the use of the prototype procedure in the prior art impossible for drug manufacturing according to GMP standards.

Starting porphyrin derivatives for the syntheses of interest are traditionally obtained from pure and standard raw porphyrin materials methyl (3) or ethyl (4) pheophorbide a. General methods known to date for the separation of porphyrins from biological raw materials consist of a long sequence of laborious washings with organic solvents and/or freezing steps to destroy cell walls of the biomaterial, and repeatable extractions together with chemical treatments of the biomass to obtain chlorophyll first, which then is transformed into pheophytin and subsequently hydrolyzed to yield pheophorbide (K. M. Smith, D. A. Goff and D. J. Simpson, J. Amer. Chem. Soc., 1985, 107, 4946-4954; R. K. Pandey, D. A. Bellnier, K. M. Smith and T. J. Dougherty, Photochem. Photobiol., 1991, 53, 65-72; W. A. Svec, In: The porphyrins, ed. D. Dolphyn, NY, Academic Press, 1978, 5, 342-400).

There is an obvious need to provide an easy and efficient method for the preparation of pure and chemically stable water-soluble porphyrin derivative drug products with a standard content of the main water-soluble porphyrin derivative drug substance, suitable for medical applications especially in photodynamic therapy. The present invention fills this need as well as other related advantages.

### SUMMARY OF THE INVENTION

It is an object of the present invention to provide chemically stable water-soluble porphyrin derivative drug products with a standard content of the water soluble porphyrin derivative drug substance and suitable for various medical applications, particularly for PDT.

It is another object of the present invention to provide a method to prepare said chemically stable water-soluble porphyrin derivatives.

Yet another object of the present invention is to provide an easy and efficient method to prepare chemically stable water-soluble porphyrin derivatives from biological raw materials avoiding the above mentioned disadvantages.

It is another object of the present Invention to provide said chemically stable water-soluble porphyrin derivatives as a drug substance preparation to necrotize and/or oxidize cancerous and hyperproliferative tissue for use in medical applications like treatment of cancer, other hyperproliferative diseases, infections and others.

In one embodiment the present invention provides a method to prepare water-soluble porphyrin derivatives for use as drug substances, and in prepared drug products, comprising the steps of a direct acidic alcoholysis, which might be done in one or two steps, of biological raw material giving crystalline alkyl pheophorbide, conversion of the obtained alkyl pheophorbide into an acidic porphyrin, interaction of the acidic porphyrin in water or in aqueous organic solution with a hydrophilic organic amine. In another embodiment the present invention provides a method to prepare water-soluble porphyrin derivatives, by a step of interaction of the acidic porphyrin in water or in aqueous organic solution with a hydrophilic organic amine. In yet another embodiment the present invention provides a method to prepare water-soluble porphyrin derivatives, comprising the steps of a direct acidic alcoholysis, which might be done in one or two steps, of biological raw material giving crystalline alkyl pheophorbide, conversion of the obtained alkyl pheophorbide into an acidic porphyrin, interaction of the acidic porphyrin in water or in aqueous organic solution with a hydrophilic organic amine and purification of a water-soluble porphyrin derivative by reversed phase chromatography using of volatile solvents. In still another embodiment, the present invention provides a method to prepare water-soluble porphyrin derivatives, comprising the steps of interaction of the acidic porphyrin in water or in aqueous organic solution with a hydrophilic organic amine, and purification of a water-soluble porphyrin derivative by reversed phase chromatography with the use of volatile solvents. Furthermore, the present invention provides water-soluble porphyrin derivatives of formula (1), useful for medical applications, obtained by the methods provided by the invention.

B is a ring having the structure:

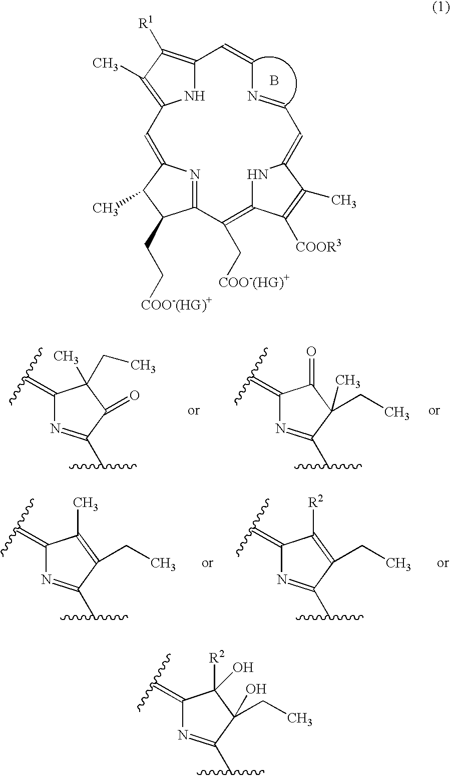

where:

R¹=—CH═CH₂, —CH(OAlk)CH₃, —CHO, —C(O)CH₃; —CH₂CH₃; —CH(Alk)CH(COAlk)₂, —CH₂CH(COAlk)₂, —CH(Alk)CH₂COAlk, —CH(Alk)CH₂CH(OH)CH₃, —CH₂CH₂CH(OH)CH₃

R²=—CH₃, —CHO, —CH(OH)Alk, —CH═CHAlk, CH₂OH, CH₂OAlk;

R³=H or lower alkyl;

G=hydrophilic organic amine (e.g. N-methyl-D-glucamine and other amino-group containing carbohydrate derivatives, TRIS, aminoacids, oligopeptides).

Alk=alkyl substituent.

The above, and other objects, features and advantages of the present invention will become apparent from the following description read in conjunction with the accompanying drawings.

### BRIEF DESCRIPTION OF THE DRAWINGS

FIG. 1: Determination of dark toxicity (cytotoxicity, Example 14) of water-soluble salt of chlorin e₆ (5) with N-methyl-D-glucamine (8) being prepared (A) according to this invention (Example 9) and (B) according to Ponomarev (RU2144538); the test was performed in OV2774 cells under addition of different concentrations of the photosensitizer as indicated.

FIG. 2: Determination of phototoxicity (Example 15) of water-soluble salt of chlorin e₆ (5) with N-methyl-D-glucamine (8) being prepared (A) according to this invention (Example 9) and (B) according to Ponomarev (RU2144538); the test was performed in OV2774 cells under addition of different concentrations of the photosensitizer as indicated and irradiation at 670 nm.

### DETAILED DESCRIPTION OF THE PREFERRED EMBODIMENTS

Prior to setting forth the invention it may be helpful to set forth definitions of certain terms to be used within the disclosure.

Porphyrins are macrocycle compounds with bridges of one carbon atom or one nitrogen atom respectively, joining the pyrroles to form the characteristic tetrapyrrole ring structure. There are many different classes of porphyrin derivatives including that ones containing dihydro-pyrrole units. The term porphyrins will be used herein to refer to porphyrins, phthalocyanines, chlorins, metallo derivatives thereof and other porphyrin-like compounds suitable for PDT and pharmaceutical preparations.

As used herein, biological raw materials are materials for preparation of compounds of the present invention, comprising e.g. plants, algae, blood components, insect excretions.

Also, as used herein, “drug substance” is used as defined by the United States Food and Drug Administration. according to 21 C.F.R. 314.3, a “Drug substance means an active ingredient that is intended to furnish pharmacological activity or other direct effect in the diagnosis, cure, mitigation, treatment, or prevention of disease or to affect the structure or any function of the human body, but does not include intermediates use in the synthesis of such ingredient.”

Further, “drug product” is used herein as defined by the United States Food and Drug Administration according to 21 C.F.R. 314.3, which defines a drug product as “a finished dosage form, for example, tablet, capsule, or solution, that contains a drug substance, generally, but not necessarily, in association with one or more other ingredients.”

The aim of the invention is achieved by the proposed method, comprising the interaction of acidic porphyrins in water or aqueous organic solution with a hydrophilic organic amine preferably with N-methyl-D-glucamine (8) which is a polyhydroxylated stable and non-toxic compound useful for drug preparation or with amino alkyl and amino aryl glycosides, for example maltose derivatives (9) and (10), or other amino-group containing carbohydrate derivatives. Other proposed reagents are e.g. TRIS, tris(hydroxymethyl)aminomethane (11) which is also a stable and non-toxic compound useful for drug preparation, or with TRIS derivatives, for example compounds (12) and (13), as well as other types of hydrophilic amines, for example bis(2-hydroxyethyl)amine (14). Aminoacids or oligopeptides for example oligolysines, preferentially penta- and hexa-lysines also can be used as hydrophilic organic amines suitable for preparation of water-soluble porphyrin derivatives of the present invention.

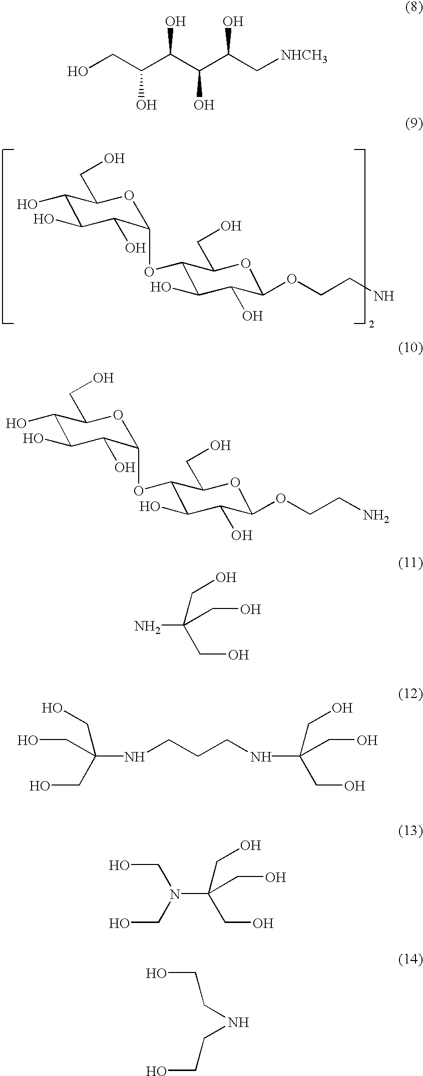

According to the first preferred embodiment of the present invention, as described in Example 9, the quantitative stoichiometric interaction of chemically pure porphyrin (as free acid) and an appropriate hydrophilic organic amine is performed at room temperature under inert gas and in darkness. The solvent used is either chemically pure water which is degassed with an inert gas (e.g. argon, helium or others), or, if necessary, a mixture of water with a suitable chemically pure and degassed organic solvent. The organic solvent is subsequently evaporated in vacuo without heating (to avoid possible destruction of starting porphyrin), and the product is freeze-dried. In some cases it is necessary to add an organic solvent to assist the reaction by dissolving the starting porphyrin so that the interaction of the porphyrin (as free acid) and appropriate hydrophilic organic amine takes place. Examples for possible organic solvents are acetone or a mixture of methylene chloride and methanol. The resulting freeze-dried water-soluble porphyrin is chemically pure and does not need any further purification except sterilization for medical or biological applications.

According to another preferred embodiment of the present invention, impure ingredients can be used including wet pastes of starting porphyrins. The reaction is performed similarly as described above, but an excess amount of hydrophilic organic amine is used to involve all porphyrin components in the reaction. After the concentration of reaction mixture in vacuo the resulting water-soluble porphyrin is purified by the chromatography on the column with appropriate reversed phase adsorbent, preferentially of RP C-8 or C-18 types. Fractions with target products are collected, evaporated in vacuo without heating to remove organic solvent, and freeze-dried to give the desired water-soluble porphyrin derivative. Developed protocols for purification of water-soluble porphyrin derivatives by reversed phase chromatography with the use of volatile solvents yield the high quality which is critical in the manufacture of medical preparations.

In another embodiment the present invention is an easy and efficient method of obtaining porphyrin compounds from biological raw materials. The method comprises undergoing a direct acidic alcoholysis, which might be done in a one or two steps, of biological raw materials, preferably methanolysis or ethanolysis, giving crystalline alkyl pheophorbides (preferably methyl and ethyl) as key intermediate products suitable for a variety of further chemical transformations to obtain the target porphyrin derivatives. This simple and efficient procedure permits the preparation of porphyrin derivatives from biological raw materials without the use of laborious washings with organic solvents or freezing (to destroy cell walls) of the starting biomaterials and repeatable extractions as necessary in the known procedures.

Application of crystalline alkyl pheophorbides (preferably methyl and ethyl) as synthetic intermediate products allows for simple purification and standardization that is critical for manufacturing medical preparations.

Performance of alcoholysis depends on the quality of starting biological raw material and particularly on its dryness which is important for maintaining the necessary acid concentration during alcoholysis. Thus, in the case of sufficiently dry material, e.g. dried Spirulina or Chlorella biomasses or powdered dry nettle leaves (see Examples 1-5) it is possible to perform direct one step preparation of alkyl pheophorbides.

In the case of insufficiently dry raw material, the preparation of alkyl pheophorbides is performed by a two step alcoholysis as exemplified by the preparation of methyl pheophorbides a (3) and b (15) from spinach (see Example 6). In such cases the presence of excessive amounts of water in the starting raw material does not permit reaching the appropriate concentration of acid suitable to perform the cleavage of the phytol ester. Nevertheless, pheophytins, being obtained after the first alcoholysis step, are dry enough to be used in the preparation of crystalline alkyl pheophorbides within the second alcoholysis step.

Preparation of acidic porphyrins suitable for further transformation into water-soluble forms is performed by chemical transformation of porphyrin raw materials. For example, crystalline alkyl pheophorbides obtained from biological raw materials according to the procedure described in the present invention.

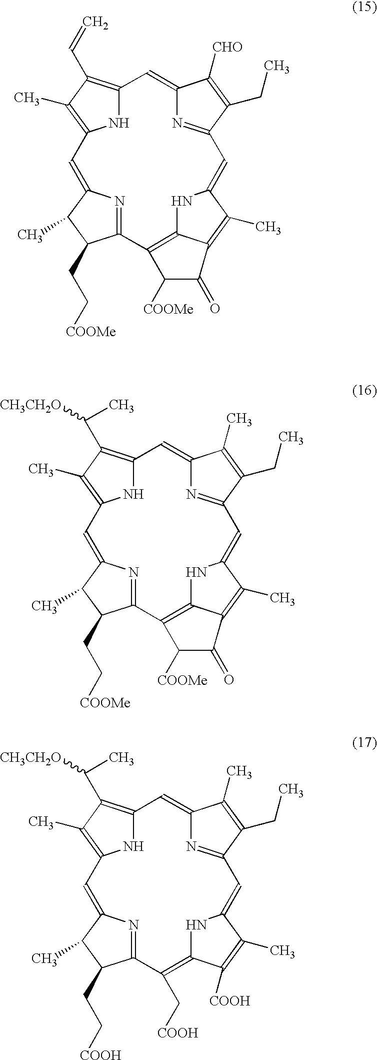

Particularly 2-devinyl-2-(1-alkoxyethyl)-chlorins e₆, e.g. ethoxy-derivative (17), could be obtained by hydrobromination of methyl or ethyl pheophorbides a with saturated solution of HBr in acetic acid to give respective 2-devinyl-2-(1-bromoethyl)-pheophorbides a, their further alcoholysis affording to 2-devinyl-2-(1-alkoxyethyl)-pheophorbides a (e.g. compound 16) (C. Rimington, A. Roennestad, A. Western, and J. Moan, Int. J. Biochem., 1988, 20, 1139-1149; K. R. Adams, C. R. Berembaum, R. Bonnett, A. N. Nizhnik, A. Salgado, and M. A. Valles, J. Chem. Soc. Perkin Trans. 1, 1992, 1465-1470) and subsequent saponification with formation of respective 2-devinyl-2-(1-alkoxyethyl)-chlorins e₆ as triacids (17) or water-soluble salts, e.g. with N-methyl-D-glucamine (8) (Example 10).

In a similar manner the interaction of other types of nucleophiles with 2-devinyl-2-(1-bromoethyl)-pheophorbides instead of their alcoholysis affords the formation of a variety of possible porphyrin derivatives to be used in the preparation of their water-soluble forms according to the present invention.

The interaction of chlorins with hydrophilic amines according to the present invention leads to the formation of mainly of bis-salts, because the carboxyl group at position 6 (atom numbering is shown for compounds 2-4) does not exhibit sufficient acidity for salt formation. Due to the reversible character of the reaction of bis-salt formation (Scheme 1) it is desirable to perform it in the presence of the excess of hydrophilic amine.

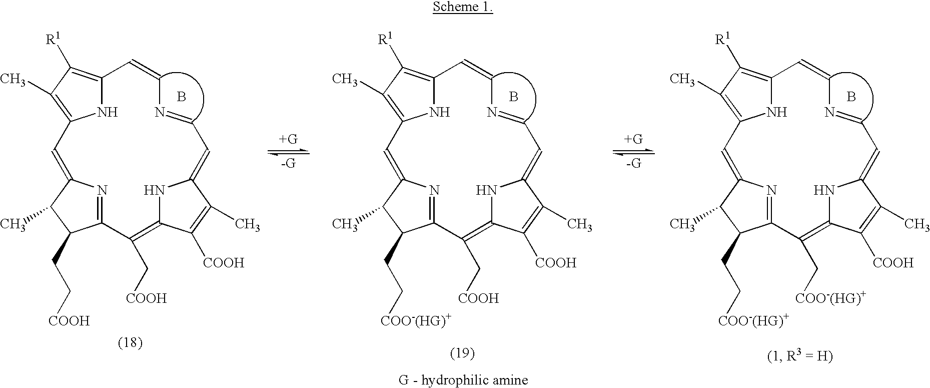

Water-soluble chlorin bis-salts of present invention can be obtained in the individual state by column chromatography purification as exemplified in Example 9B. Dissolving of purified bis-salts in water leads to reversible hydrolysis to give mono-salts (e.g. of type 19, see Scheme 2) which may have reduced water solubility compared to bis-salts and, probably, the parent chlorin (1) which is poorly soluble in water. Such processes can result in the formation of small precipitate during the storage of solutions.

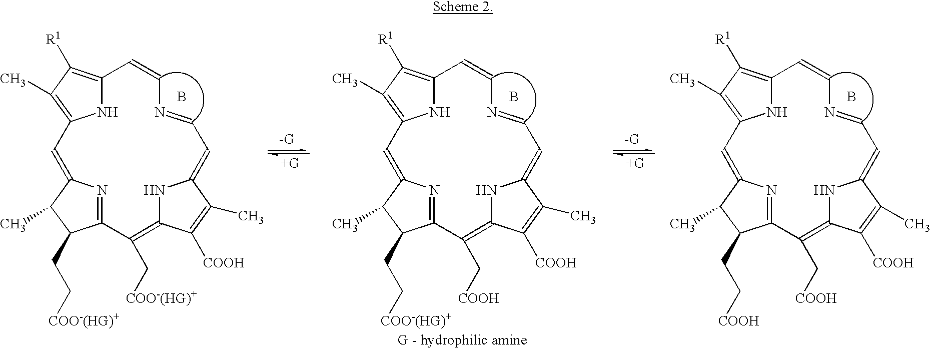

To prevent such undesirable processes and maintaining clear and homogeneous solutions, which is strictly necessary for their usage in medical applications, e.g. in the field of PDT, it is preferential to dissolve bis-salts in water in the presence of small and known amount of the respective hydrophilic amine (e.g. lower than 2 mole equivalents, more preferably between 0.1 and 0.5 equivalents), so that the desired porphyrin derivatives are kept in the form of bis-salts and thus the formation of mono-salts and parent chlorins by hydrolysis of bis-salts is prevented.

It is another object of the present invention to use the chemically stable and water-soluble porphyrin derivatives according to formula (1) as drug substances for various medical applications. Said compounds are especially preferable for the use in PDT to oxidize and/or necrotize cancerous tissue, and for the treatment of other hyperproliferative diseases, infections and other diseases. Due to the water-solubility said compounds are prepared in various pharmaceutically acceptable and active drug products for different administration methods, e.g. injections.

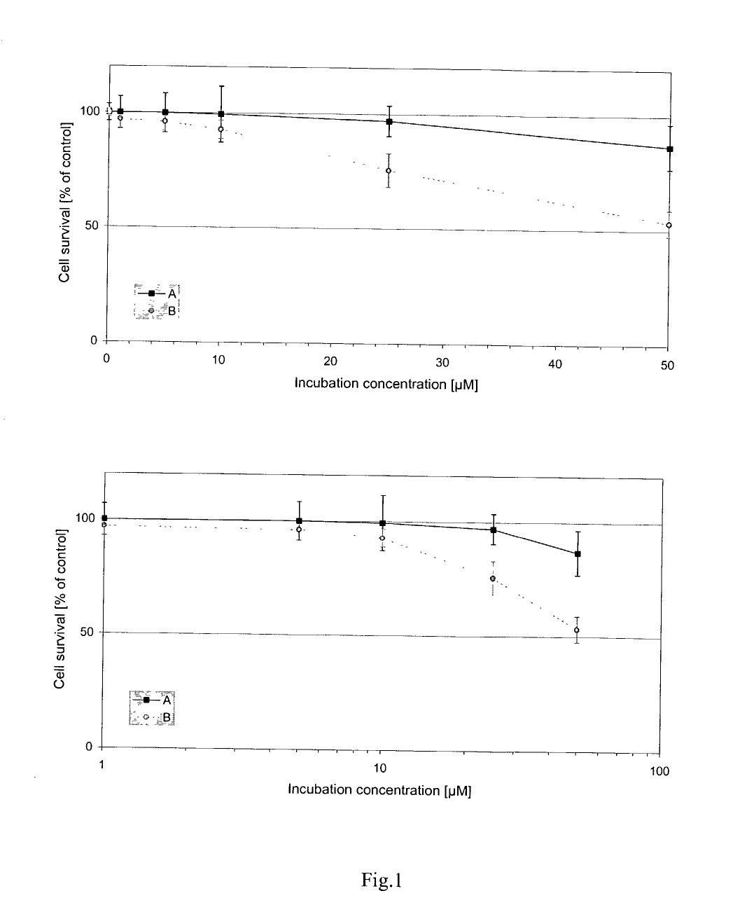

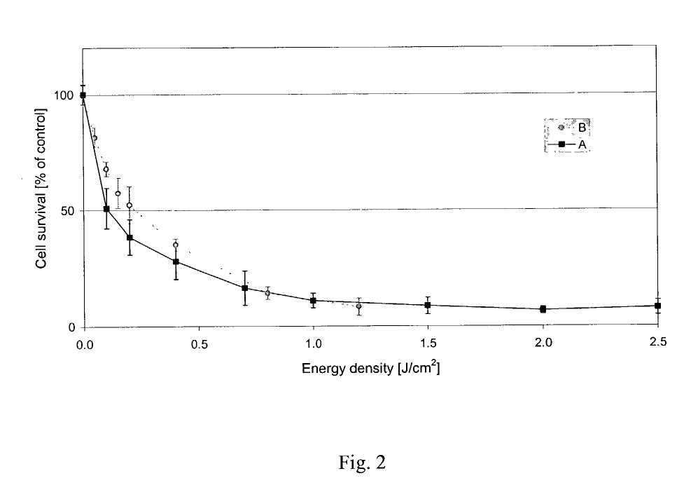

Determination of dark toxicity (example 14, FIG. 1) and photo toxicity (example 15, FIG. 2) of one of the porphyrin derivatives of the present invention, namely the water-soluble salt of chlorin e₆ (5) with N-methyl-D-glucamine (8) being prepared according to Example 9 in cell culture experiments showed excellent properties of the compounds for the use in PDT. The experiments were carried out to also demonstrate the inferior characteristics (higher dark toxicity and lower photo toxicity) of a same compound but being prepared according to the Ponomarev technology (RU2144538).

However, in special cases the administration of defined mixtures of hydrophilic amine salts of different porphyrin derivatives might be advantageous, if these mixtures display a higher photo toxicity towards the diseased tissue. This enhanced photo toxicity might be caused by the observed phenomenon of the enhancement of water solubility of the mixture of compounds (2), (5) and (6) as salts with hydrophilic amines in the presence of the same salts of chlorin e₆.

### EXAMPLES

The following examples are put forth so as to provide those of ordinary skill in the art with a complete disclosure and description of how to make the water-soluble porphyrin derivatives of the invention to be used as drug substances in suitable drug products and are not intended to limit the scope of the invention. Efforts have been made to ensure accuracy with respect to numbers used (e.g. amounts, temperature etc.), but some experimental errors and deviations should be accounted for.

### Example 1

Obtaining Methyl Pheophorbide (3) from Spirulina platensis

(A) A mixture of 20 g of Spirulina platensis, 60 mL of methanol and 10 mL of concentrated sulfuric acid was stirred at room temperature for 3 hours, diluted with 30 mL of methanol and filtered through a pad of Celite. The content of filtrating funnel was washed with methanol (70 mL). The above solution was extracted with hexane (2×30 mL), diluted with chloroform (100 mL) and poured into saturated aqueous solution of potassium chloride (300 mL). The resulting mixture was filtered through a pad of Celite, then the aqueous phase was extracted with chloroform (2×50 mL). The combined extracts were washed with water, filtered through cotton and concentrated. The residue was dissolved in the mixture of chloroform-hexane (1:1, 30 mL) and filtrated through a pad of aluminum oxide to wash first with hexane (to remove non-polar non-chlorin components) and then with methylene chloride (to get methyl pheophorbide). Methylene chloride solution was concentrated and the residue was re-crystallized first from methylene chloride-methanol (3 mL+7 mL), and then from methylene chloride-methanol (1 mL+10 mL), and finally washed with methanol (10 mL) to give 113 mg of pure methyl pheophorbide a (3). ¹H-NMR spectrum: 9.41, 9.23, 8.56 (3H, all s, meso-H); 7.88 (1H, q, —CH═CH₂), 6.25-6.10 (2H, —CH═CH₂) 6.28 (1H, s, cyclopentanone-H), 4.50, 4.25 (2H, m, 7-H, 8-H); 3.55 (2H, q, 4-CH₂CH₃); 3.93, 3.70, 3.63, 3.39, 3.15 (15H, all s, 5×—CH₃); 2.75-2.20 (4H, m, —CH₂CH₂COOCH₃); 1.85 (3H, d, 8-CH₃); 1.71 (3H, m, 4-CH₂CH₃); 0.55 and −1.68 ppm (2H, 2 broad s, 2×—NH—). The product is identical to the same compound obtained from Porphyrin Products Inc., USA.

(B) A mixture of 10 g of Spirulina platensis, 30 mL of methanol, and 5 mL of concentrated sulfuric acid was stirred at room temperature (r.t.) for 3 h, diluted with cold water (70 mL), and filtered through a pad of Celite. The content of filtrating funnel was washed with water up to pH 7, ethanol (50 mL), hexane (4×30 mL), and the required methyl pheophorbide was removed from the content of filtrating funnel with acetone (120 mL). The above solution was concentrated, dissolved in chloroform, filtered through a pad of sodium sulfate (anhydrous), and concentrated. The residue was re-crystallized first time from methylene chloride-methanol (1.5 mL+5 mL), second time from methylene chloride-methanol (1.5 mL+10 mL), and finally washed with methanol (15 mL) to give 60 mg of pure methyl pheophorbide a (3).

(C) To a suspension of 500 g of Spirulina platensis in MeOH (1500 ml), H₂SO₄ (conc., 250 ml) was added at room temperature and under stirring. The mixture formed was kept at r.t. for 3 h, then poured into water (6 L) and filtered through a pad of Celite (d 12 cm, h 2 cm; on filter Schott N3). The paste on filter was washed with water (3×800 ml, until pH 6), then with ethanol (3×300 ml) and petroleum ether (40-60° C., 3×250 ml). After that the target product was removed from the pad with acetone (in total 1.2 L), concentrated, dissolved in CHCl₃ (200 ml), filtered through a cotton wool, concentrated in vacuo and the residue was crystallized from a mixture of CH₂Cl₂ (40 ml) and MeOH (200 ml) to give 2.45 g of methyl pheophorbide a of ˜90% purity (TLC in CHCl₃/acetone 95:5). The latter was dissolved in CHCl₃ (20 mL) and passed through a pad of Al₂O₃ (neutral, Grade II; d 8 cm, h 5 cm) by elution with CHCl₃; concentration and re-crystallization from a mixture of CH₂Cl₂ (50 ml) and MeOH (250 ml) gave 2.38 g of pure (TLC control) methyl pheophorbide a (3). Additional amounts of methyl pheophorbide a (˜10-15%) can be obtained by usual workup (chromatography and re-crystallization) of mother solutions from both crystallizations.

### Example 2

Obtaining Ethyl Pheophorbide (4) from Spirulina platensis

Ethanolysis of 20 g of Spirulina platensis in 60 ml of 96% aqueous ethanol and 10 ml concentrated sulfuric acid and subsequent workup as described in the Example 1A for the preparation of methyl pheophorbide a (3) but with the use of ethanol instead of methanol in all steps, gave 110 mg of crystalline ethyl pheophorbide a (4). ¹H-NMR spectrum: 9.57, 9.42, 8.61 (3H, all s, meso-H); 7.99 (1H, q, —CH═CH₂), 6.32, 6.26 (2H, dd, —CH═CH₂), 6.27 (1H, s, cyclopentanone-H), 4.51, 4.28 (2H, m, 7-H, 8-H); 4.07 (2H, q, —COOCH₂CH₃); 3.71 (2H, q, 4-CH₂CH₃); 3.89, 3.72, 3.41, 3.27 (12H, all s, 4×—CH₃); 2.69, 2.47, 2.37, 2.22 (4H, m, —CH₂CH₂COOCH₂CH₃); 1.83 (3H, d, 8-CH₃); 1.73 (3H, t, 4-CH₂CH₃); 1.12 (3H, t, —COOCH₂CH₃; 0.57, −1.46 ppm (2H, 2 broad s, 2×—NH—).

### Example 3

Obtaining Methyl Pheophorbide a (3) from Spirulina maxima

A treatment of 10 g of Spirulina maxima with 30 mL of methanol and 5 mL of concentrated sulfuric acid and subsequent workups as described for preparation of methyl pheophorbide a (3) in the Example 1B afforded to 64 mg of pure methyl pheophorbide a (3).

### Example 4

Obtaining Methyl Pheophorbide a (3) and b (15) from Chlorella

10 g of dry biomass of Chlorella was subjected to methanolysis and subsequent workups as described in the Example 3 to give the mixture (140 mg) of methyl pheophorbides a and b in the ratio 20:7 as determined by ¹H NMR spectroscopy. Chromatography of the mixture obtained on the column with Silica gel 60 (Fluka, 70-230 mesh) with the elution chloroform-toluene-acetone 15:30:1.5 gave individual methyl pheophorbides a (3) and b (15). Methyl pheophorbides a (3) was identical to described above products. ¹H-NMR spectral data for methyl pheophorbide b (15): 11.0 (1H, s, CHO), 10.22, 9.50, 8.55 (3H, all s, meso-H); 7.98 (1H, q, —CH═CH₂), 6.40-6.15 (2H, —CH═CH₂), 6.25 (1H, s, cyclopentanone-H), 4.48, 4.20 (2H, m, 7-H, 8-H); 3.60 (2H, q, 4-CH₂CH₃); 3.93, 3.78, 3.75, 3.40 (12H, all s, 4×—CH₃); 2.75-2.20 (4H, m, —CH₂CH₂COOCH₃); 1.85 (3H, d, 8-CH₃); 1.71 (3H, m, 4-CH₂CH₃); 0.48 and −1.60 ppm (2H, 2 broad s, 2×—NH—).

### Example 5

Obtaining Methyl Pheophorbide a and b from Powdered Dry Nettle Leaves

Methanolysis of 500 g dried and powdered nettle leaves and subsequent workups as described in the Example 1C afforded to the mixture (1.74 g) of methyl pheophorbides a (3) and b (15) in the ratio 6.5:1 as determined by ¹H NMR spectroscopy.

### Example 6

Obtaining Methyl Pheophorbide a and b from Frozen Spinach Leaves

To the mixture of 100 g frozen spinach leaves and MeOH (100 ml), H₂SO₄ (conc., 5 ml) was added at r.t. and under stirring. The mixture formed was kept at r.t. 16 h, diluted with water (100 ml), and filtered through Celite. The residue was washed with acetone (3×50 ml), acetone extracts were diluted with CH₂Cl₂-water (1:1, 100 ml), organic phase was separated and concentrated. The residue was subjected to methanolysis in 100 ml of 5% conc. H₂SO₄ in MeOH and subsequent workups as described in the Example 1C gave 40 mg of the mixture of methyl pheophorbides a (3) and b (15) in the ratio 2:1 as determined by ¹H NMR spectroscopy.

### Example 7

Preparation methyl 2-devinyl-2-(1-ethoxyethyl)-pheophorbide a (16)

Methyl pheophorbide a (3) 3.5 g (5.8 mmol) is dissolved in the mixture of hydrogen bromide and acetic acid (d 1.44, 50 ml) and left for 18 h. The mixture is then evaporated to dryness at 50° C. in vacuo, and absolute ethanol (100 ml) is added under stirring. After 18 h, the reaction mixture was poured onto crashed ice under stirring and extracted with CH₂Cl₂ (3×40 ml). The combined extract was washed with water (4×70 ml) and evaporated to dryness in vacuo. The residue was subjected to column chromatography on silica gel (40-63 μm, Merck) with eluting by CH₂Cl₂ to give 2.95 g of product (16), yield 77%. ¹H-NMR spectrum: 9.82, 9.58, 8.55 (3H, all s, meso-H); 6.31 (1H, s, 10-H), 5.96 (1H, q, 2-CHCH₃); 4.53, 4.25 (2H, m, 7-H, 8-H); 3.71 (4H, dq, 4-CH₂CH₃, —OCHhd 2CH₃); 3.93, 3.87, 3.63, 3.41, 3.28 (15H, all s, 5×—CH₃); 2.67, 2.51, 2.37, 2.23 (4H, m, —CH₂CH₂COOCH₃); 2.11 (3H, d, 2-CHCH₃); 1.80 (3H, d, 8-CH₃); 1.75 (3H, t, 4-CH₂CH₃); 1.36 (3H, t, —OCH₂CH₃); 0.55, −1.41 (2H, 2 broad s, 2×—NH).

### Example 8

Preparation of Chlorin e₆ (5)

To a stirred solution of 140 mg (231 μmoles) methyl pheophorbide a (3) in degassed (with helium) acetone (12 ml) under argon, an aqueous (aq.) solution of KOH (degassed, 10%-soln, 10 ml) was added. The mixture was stirred 40 min under 40° C., heated up to 65 ° C. and 3% aq. solution of KOH (prepared from 5 ml of degassed 10% aq. KOH and 11 ml of degassed water) was added. The resulting mixture was stirred for 2.5 h under argon and heating at 65° C., cooled to r.t., diluted with 100 ml of water, acidified with 2 N HCl (12 ml). The precipitate was separated by centrifugation (3 min at 5000 rpm), washed with water (3×30 ml) with re-centrifugation, re-suspended in 10 ml of water and freeze dried to give crude chlorin e6 (120 mg, 87%, ˜90% purity, controlled with TLC) as free acid. TLC: RP-18 TLC plates (Merck), MeOH—CH₂Cl₂ (3:1), Rf 0.6; contaminants: more polar impurities with Rf<0.3. Final purification: 20 mg of crude chlorin e6 was dissolved in MeOH—CH₂Cl₂-water (3:1:1, 4 ml) and subjected to MPLC on RP-8 column (Merck, #11447, 240×10, 40-63 μm) with elution by MeOH—CH₂Cl₂-water (4:3:1, 2 ml/min) to give pure chlorin e6 (15 mg, 75%). A contaminant impurity was eluted with MeOH—CH₂Cl₂ (3:1). ¹H-NMR spectrum (DMSO-d₆): 9.88, 9.78, 9.18 (3H, all s, meso-H); 8.33 (1H, dd, —CH═CH₂); 6.47 (1H, d, cis —CH═CH₂); 6.22 (1H, d, trans —CH═CH₂); 5.38 (2H, m, —CH₂COOH); 4.62 (1H, m, —CHCH₃); 4.48 (1H, m, —CHCH₂); 3.83 (2H, m, —CH₂CH₃); 3.59, 3.53, 3.33 (9H, all s, —CH₃); 2.62, 2.27, 2.14, (4H, m, —CH₂CH₂COOH); 1.70, 1.66 (6H, m, —CHCH₃+—CH₂CHhd 3); 1.64, −1.90 (2H, 2 broad s with different intensity, 2×—NH—). ¹³C-NMR spectrum (DMSO-d₆) characteristic signals only: 174.15, 173.46, 172.34 (COOH); 129.21 (—CH═CH₂); 122.25 (—CH═CH₂); 103.74 (γ); 101.12 (β); 98.09 (α); 94.67 (δ); 52.66 (CHCH₂); 48.19 (CHCH₃); 37.80 (—CH₂COOH); 30.82, 29.50 (—CHCH₂CH₂COOH); 22.92 (CHCH₃); 18.91 (CH₂CH₃); 17.58 (CH₂CH₃); 11.00, 10.98 (ArMe).

### Example 9

Preparation of Water-soluble Salt of Chlorin e₆ (5) with N-methyl-D-glucamine (8)

(A) To a solution of 5 mg (8.4 μmol) chlorin e₆ (5) in MeOH—CH₂Cl₂ (3:1, 20 mL) (or in acetone) a solution of (4 mg) (21 ,μmol) N-methyl-D-glucamine (8) in water (4 mL) was added and organic solvents were evaporated off in vacuo. Resulting aqueous solution was filtered through membrane (20 μm) and freeze dried to give the water-soluble salt (9 mg, includes the excess of N-methyl-D-glucamine) quantitatively.

(B) To a solution of 50 mg of methyl pheophorbide a (3) in degassed acetone (12 mL) 10% aqueous degassed solution of potassium hydroxide (10 mL) was added. The mixture was stirred at 40° C. for 30 minutes under inert atmosphere (argon) followed by addition of 15 mL of 3% aqueous degassed solution of potassium hydroxide. The resulting mixture was stirred at 65° C. for 2 hours under inert atmosphere (argon), diluted with water (100 mL), and chlorine e₆ (5) was precipitated by addition of 2N aqueous solution of HCl (up to pH 6). The precipitate was separated by centrifugation (3000 rpm for 5 minutes), washed with water (3×10 mL) to give wet paste of chlorin e₆ which was used in the next step directly, without additional purification. The whole sample of thus obtained chlorin e₆ (5) was mixed under argon with N-methyl-D-glucamine (8) (30 mg., 2 eq.) and water (10 mL) to get about 5% solution of the water-soluble salt. The resulting mixture was stirred until complete dissolving, evaporated to dryness and subjected further HPLC on the column with RP C-8 in water-methanol gradient (from 40% to 80%). Fractions with target product were collected and lyophilized to give water-soluble salt in about 75-80% yield.

(C). The mixture of 50 mg (84 μmol) of powdered chlorin e₆ (5), 40 mg (0.21 mmol) N-methyl-D-glucamine (8) and water (50 ml, preliminary degassed with inert gas) was stirred under argon and in darkness until complete dissolving. Resulting solution was filtered through membrane (20 μm) and freeze dried to give the water-soluble salt (90 mg, includes the excess of N-methyl-D-glucamine) quantitatively.

### Example 10

Preparation of 2-devinyl-2-(1-ethoxyethyl)-chlorin e6 (17).

Methyl 2-devinyl-2-(1-ethoxyethyl)-pheophorbide a (3) (2.8 g, 4.1 mmol) was subjected to saponification as described for preparation of chlorin e6 (Example 8) to give after column chromatography 2.1 g of product (17), yield 79%. ¹H-NMR spectrum (DMSO-d₆): 9.88, 9.78, 9.18 (3H, all s, meso-H); 6.47 (1H, d, cis —CH═CH₂); 6.22 (1H, d, trans —CH═CH₂); 5.5.1 (2H, m, —OCH₂CH₃); 5.38 (2H, m, —CH₂COOH); 4.62 (1H, m, —CHCH₃); 4.48 (1H, m, —CHCH₂); 4.29 (1H, q, —CHCH₃); 3.83 (2H, m, —CH₂CH₃); 3.59, 3.53, 3.33 (9H, all s, —CH₃); 2.62, 2.27, 2.14, (4H, m, —CH₂CH₂COOH); 1.83 (1H, d, —CH(O—)CH₃); 1.70, 1.66 (6H, m, —CHCH₃+—CH₂CH₃); 1.54 (3H, t, —OCH₂CH₃); −1.90 (2H, 2 broad s with different intensity, 2×—NH—). ¹³C-NMR spectrum (DMSO-d₆) characteristic signals only: 174.15, 173.46, 172.34 (COOH); 129.21 (—CH═CH₂); 103.74 (γ); 101.12 (β); 98.09 (α); 94.67 (δ); 67.97 (—CH (O—)CH₃); 63.49 (—OCH₂CH₃); 52.66 (CHCH₂); 48.19 (CHCH₃); 37.80 (—CH₂COOH); 30.82, 29.50 (—CHCH₂CH₂COOH); 22.92 (CHCH₃); 20.20 (—CH(O—)CH₃); 18.91 (CH₂CH₃); 17.58, 15.23 (CH₂CH₃+—OCH₂CH₃); 11.00, 10.98 (ArMe).

### Example 11

Preparation of Water-soluble Salt of Chlorin e₆ Derivative (17) with N-methyl-D-glucamine (8)

Interaction of 30 mg of 2-devinyl-2-(1-etoxyethyl)-chlorin e₆ (17) with 20 mg of N-methyl-D-glucamine (8) as described in Example 9C gave 50 mg of freeze dried water-soluble salt quantitatively.

### Example 12

Preparation of bis[2-(β-maltosyloxy)ethyl]amine (9)

To a stirred mixture of silver trifluoromethanesulfonate (208 mg, 0.805 mmol) and 1.5 ml of absolute CH₂Cl₂ at −20° C., a solution of per-O-acetyl-maltosylbromide (500 mg, 0.7 mmol), N-benzyloxycarbonyl-N,N-bis(2-hydroxyethyl)amine (56 mg, 0.233 mmol) and 2,4,6-trimethylpyridine (80 1 μl, 0.605 mmol) was added drop wise. The mixture was left to warm to r.t., treated with 0.5 ml of triethylamine, diluted with 200 ml of dichloromethane, washed with saturated aqueous solution of Na₂S₂O₃ (50 ml) and water (50 ml), concentrated and subjected to flash chromatography in ethyl acetate—petroleum ether (1:1) to give crude N-benzyloxycarbonyl-N,N-bis[2-(hepta-O-acetyl-β-maltosyloxy)ethyl]amine (57 mg). Selected structure specific ¹³C NMR data (500 MHz, CDCl₃): 20.41 (CH₃CO); 46.92, 47.24, 48.00 and 48.02 (OCH₂CH₂N and OCH₂CH₂N); 61.30, 61.50, 61.98, and 62.52 (C-6 of glucose moieties); 67.18 (NCOOCH₂C₆H₅); 67,81, 68,33, 69.14, 69.84, 72.02, 72.55, 75.10 (C-2-C-5 of glucose moieties); 95.38 (C-1 of α-glucose moieties); 100.18 and 101.10 (C-1 of β-glucose moieties); 127.73, 128.01 and 128.40 (OCH₂C₆H₅); 164.00 (NCOO); 169,24, 169.42, 169.76, 169.98, 170.35 (CH₃CO). [α]^{D} 38.9° (c 1, ethyl acetate). The product obtained was dissolved in 0.1 M sodium methylate in absolute methanol and kept for 2 h., then was neutralized with ion-exchange resin KU-2(H⁺) and filtered. The filtrate was subjected to hydrogenolysis under Pd/C overnight, filtered, and freeze dried to yield 23 mg of compound (9), [α]^{D} 71° (c 1, water). Selected structure specific ¹³C NMR data (500 MHz, D₂O): 48.74 (OCH₂CH₂N), 50.85 (OCH₂CH₂N); 61.72 (C-6 of α-glucose moieties), 61.92 (C-6 of β-glucose moieties), 77.28 (C-4 of β-glucose moieties); 100.83 (C-1 of α-glucose moieties); 103.40 (C-1 of β-glucose moieties).

### Example 13

Preparation of Water-soluble Salt of Chlorin e₆ (5) with bis[2-(β-maltosyloxy)ethyl]amine (9).

Interaction of 5 mg of chlorin e₆ (5) with 18.6 mg of amine (9) as described in Example 9C gave 23 mg of freeze dried water-soluble salt almost quantitatively.

### Example 14

Determination of Dark Toxicity (Cytotoxicity, Example 14) of Water-Soluble Salt of Chlorin e₆ (5) with N-methyl-D-glucamine (8) Being Prepared (A) according to this Invention (Example 9) and (B) According to Prototype (RU2144538) in OV2774 Cells

To determine cytotoxicity (dark toxicity) of the water-soluble salt of chlorin e₆ (5) with N-methyl-D-glucamine (8) being prepared (A) according to this invention (Example 9) and (B) according to prototype (RU2144538), OV2774 cells (seeding density: 50-75 cells/mm² in RPMI-1640 w. P. (with phenol red), 5% fetal calf serum, 2 mM Glutamax I, 100 μg/ml Penicillin/Streptomycin) were incubated with increasing concentrations of up to 50 μM for 24 h. Cell survival was measured after additional 24 h in sensitizer free medium using the neutral red assay. Values are expressed as percentage of non-incubated controls. For each incubation concentration, three independent experiments were performed in quadruplicates. Data of the experiments are given in FIG. 1. Photosensitizer A showed a lower cytotoxicity towards OV2774 cells as compared with compound B. Cell survival was not significantly decreased for incubation concentrations up to 25 μM (compound B: 10 μM). So far, the IC₅₀ value (incubation concentration, which decreases cell growth to 50% as compared with controls) could not be determined. The highest tested incubation concentration resulted in a cell survival of greater 80%.

### Example 15

Determination of Photo Toxicity (Example 15) of Water-soluble Salt of Chlorin e₆ (5) with N-methyl-D-glucamine (8) Being Prepared (A) According to this Invention (Example 9) and (B) According to Prototype (RU2144538) in OV2774 Cells Under Irradiation at 670 nm.

To determine photo toxicity of water-soluble salt of chlorin e6 (5) with N-methyl-D-glucamine (8) being prepared (A) according to this invention (Example 9) and (B) according to prototype (RU2144538), OV2774 cells (seeding density: 50-75 cells/mm² in RPMI-1640 w/o P. (with phenol red), 5% fetal calf serum, 2 mM Glutamax I, 100 μg/ml Penicillin/Streptomycin) were incubated with the same concentration used in the previous experiments with photosensitizer B (10 μM, 24 h). Illumination was performed at 670 nm (10-25 mW/cm², 0.1-2.5 J/cm²) after a second incubation period in medium without sensitizer and without phenol red.. Cell survival was measured using the neutral red assay. Values are expressed as percentage of incubated, but non-illuminated controls. Five independent experiments were performed in quadruplicates. ID₅₀ values (fluence (energy density), which decreases cell growth to 50% as compared with controls) of the samples served as a quantitative measure of photo toxicity. Data of the experiments are given in FIG. 2. Results showed a somewhat higher photo toxicity of photosensitizer A as compared with compound B. The ID₅₀ value was about half that of compound B (0.1 J/cm² vs. 0.2 J/cm²).

Having described preferred embodiments of the invention with reference to the examples, it is to be understood that the invention is not limited to the precise embodiments, and that various changes and modifications may be effected therein by skilled in the art without departing from the scope or spirit of the invention as defined in the appended claims.

We claim:

1. A method to prepare water-soluble porphyrin derivative drug substances, comprising steps of
a) a direct acidic alcoholysis of biological raw material giving crystalline alkyl pheophorbide;
b) conversion of the obtained alkyl pheophorbide into an acidic porphyrin in an aqueous-based solution;
c) interaction of the acidic porphyrin in the aqueous-based solution with a hydrophilic organic amine.

2. A method of claim 1, wherein said biological raw material is selected from the group, comprising naturally occurring plants, algae, blood components, insect excretions.

3. A method of claim 2, wherein naturally occurring plants and algae comprise Spirulina Platensis, Spirulina maxima, Chlorella, nettle and spinach.

4. A method of claim 1, wherein said direct acidic alcoholysis is selected from the group: methanolysis or ethanolysis.

5. A method of claim 1, wherein said hydrophilic organic amine is selected from the group consisting of N-methyl-D-glucamine, amino alkyl glycosides, tris(hydroxymethyl)aminomethane, aminoacids and oligopeptides, and wherein said amine is preferably N-methyl-D-glucamine.

6. A method of claim 1, wherein said aqueous-based solution is a solution in water.

7. A method of claim 1, wherein said aqueous-based solution is an aqueous organic solution.

8. A method to prepare water-soluble porphyrin derivative drug substances, comprising the step of interaction of the acidic porphyrin in an aqueous-based solution with a hydrophilic organic amine.

9. A method of claim 8, wherein said hydrophilic organic amine is selected from the group consisting of N-methyl-D-glucamine, amino alkyl glycosides, tris(hydroxymethyl)aminomethane, aminoacids and oligopeptides, and wherein said amine is preferably N-methyl-D-glucamine.

10. A method to prepare water-soluble porphyrin derivative drug substances, comprising the steps of
a) a direct acidic alcoholysis of biological raw material giving crystalline alkyl pheophorbide;
b) conversion of the obtained alkyl pheophorbide into an acidic porphyrin in an aqueous-based solution;
c) interaction of the acidic porphyrin in the aqueous-based solution with a hydrophilic organic amine; and
d) purification of a water-soluble porphyrin derivative drug substance by reversed phase chromatography with the use of volatile solvents.

11. A method of claim 10, wherein said biological raw material is selected from the group, comprising naturally occurring plants, algae, blood components, insect excretions.

12. A method of claim 11, wherein naturally occurring plants and algae comprise Spirulina Platensis, Spirulina maxima, Chlorella, nettle and spinach.

13. (amended) A method of claim 10, wherein the direct acidic alcoholysis is selected from the group: methanolysis or ethanolysis.

14. A method of claim 10, wherein said hydrophilic organic amine is selected from the group consisting of N-methyl-D-glucamine, amino alkyl glycosides, tris(hydroxymethyl)aminomethane, aminoacids and oligopeptides, and wherein said amine is preferably N-methyl-D-glucamine.

15. A method to prepare water-soluble porphyrin derivative drug substances, comprising the steps of:
a) interaction of the acidic porphyrin in an aqueous-based solution with a hydrophilic organic amine,
b) purification of a water-soluble porphyrin derivative drug substance by reversed phase chromatography with the use of volatile solvents.

16. A method of claim 15, wherein said hydrophilic organic amine is selected from the group, comprising N-methyl-D-glucamine, amino alkyl glycosides, tris(hydroxymethyl)aminomethane, aminoacids and oligopeptides, and wherein said amine is preferably N-methyl-D-glucamine.

17. Water-soluble porphyrin derivative drug substances of formula 1, wherein formula 1 is: 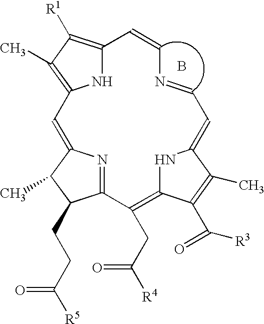
Wherein B is a ring having the structure: 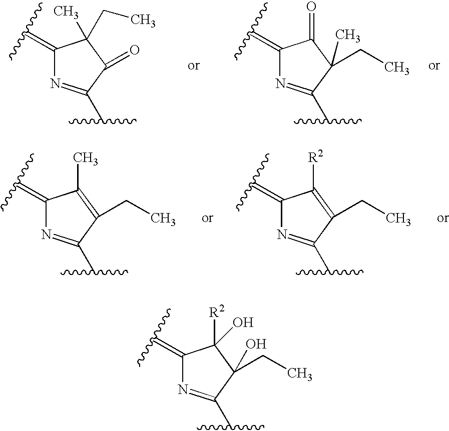
Wherein:
R¹=—CH═CH₂, —CH(OAlk)CH₃, —CHO, —C(O)CH₃, —CH₂CH₃, —CH(Alk)CH(COAlk)₂, —CH₂CH(COAlk)₂, —CH(Alk)CH₂COAlk, CH(Alk)CH₂CH(OH)CH₃, —CH₂CH₂CH(OH)CH₃;
R²=—CH₃, —CHO, —CH(OH)Alk, —CH═CHAlk, CH₂OH, and CH₂OAlk;
R³ =H or lower alkyl;
G=hydrophilic organic amine (e.g. N-methyl-D-glucamine and other amino-group containing carbohydrate derivatives, tris(hydroxymethyl)aminomethane, aminoacids, oligopeptides); and
Alk=alkyl substituent.

18. The use of said water-soluble porphyrin derivative drug substances according to claim 17 for photodynamic therapy of cancer, other hyperproliferative diseases and infections, comprising the steps of:
a) incorporating said derivatives into a pharmaceutically acceptable application vehicle;
b) administering said vehicle to a treatment area;
c) allowing for sufficient time for said porphyrin derivatives to preferentially accumulate in diseased tissue in said treatment area; and
d) irradiating said treatment area with light of a sufficient wavelength to active said porphyrin derivatives, thereby necrotizing cells of said diseased tissue.

19. Drug substances for the oxidation and/or necrotization of cancerous and other hyperproliferative tissue, wherein said water-soluble porphyrin derivatives described in claim 17 are an active ingredient.

20. Water-soluble porphyrin derivative drug substances according to formula 1, wherein said derivatives are prepared by a method selected from the group consisting of claim 1, claim 7, claim 10 and claim 15.

21. The use of said water-soluble porphyrin derivative drug substances according to claim 20 for photodynamic therapy of cancer, other hyperproliferative diseases and infections, comprising the steps of:
a) preparing a drug product by incorporating said derivatives into a pharmaceutically acceptable application vehicle;
b) administering said drug product to a treatment area;
c) allowing for sufficient time for said porphyrin derivatives to preferentially accumulate in diseased tissue in said treatment area; and
d) irradiating said treatment area with light of a sufficient wavelength to active said porphyrin derivatives, thereby necrotizing cells of said diseased tissue.

22. Drug products for the oxidation and/or necrotization of cancerous and other hyperproliferative tissue, wherein said water-soluble porphyrin derivatives described in claim 20 are an active ingredient.
